(19) United States
(12) Patent Application Publication
(10) Pub. No.: US 2010/0168590 A1
(43) Pub. Date: Jul. 1, 2010

(54) APPARATUS AND METHOD FOR ESTIMATING LIFE EXPECTANCY

(75) Inventor: Koichiro KASAMA, Kawasaki (JP)

Correspondence Address: STAAS & HALSEY LLP, SUITE 700, 1201 NEW YORK AVENUE, N.W., WASHINGTON, DC, 20005, US

(73) Assignee: Fujitsu Limited, Kawasaki (JP)

(21) Appl. No.: 12/630,100

(22) Filed: Dec. 3, 2009

(30) Foreign Application Priority Data
Dec. 26, 2008 (JP) 2008-334864

(51) Int. Cl. A61B 5/024; A61B 5/00

(52) U.S. Cl. 600/500; 600/300

(57) ABSTRACT

A life expectancy estimation apparatus for estimating a predicted value of life expectancy by pulse information of a biological body. The life expectancy estimation apparatus includes an obtaining unit operable to obtain the pulse information; a storage for storing a record of pulse information of a biological body including a plurality of entries of pulse beat rates of the biological body over a period of time; and a calculation unit operable to calculate an estimated value of life expectancy from the number of remaining pulse beats and the pulse beat rates, the number of the remaining pulse beats being estimated by subtracting the number of total pulse beats in the past estimated from the record of the pulse information from the predetermined estimated value of total pulse beats in a whole life time of the biological body.

### CROSS-REFERENCE TO RELATED APPLICATION

This application is based upon and claims the benefit of priority of the prior Japanese Patent Application No. 2008-334864, filed on Dec. 26, 2008, the entire contents of which are incorporated herein by reference.

### FIELD

The embodiments discussed herein are related to a life expectancy estimation apparatus.

### BACKGROUND

In conventional techniques for estimating the life expectancy, various types of personal data and results of health checks have been used. Accordingly, the life expectancy has been calculated by referring to the diagnosis made by medical doctors and various health-related information, but not conveniently or simply to serve health-maintaining purposes or the like.

Regarding the human health care and the like, a technique has been known which detects physiological information of a user during sleep and determines the sleeping state on the basis of the physiological information to provide health information corresponding to the sleeping state (for example, refer to Japanese Laid-open Patent Publication No. 2007-007149). According to this technique, sleeping state determining means for determining the sleeping state on the basis of the physiological information of the user during sleep is provided. In addition, health information storing means for storing the relationship between the sleeping state and the health information related to the physical and mental conditions corresponding to the sleeping state is used. The health information corresponding to the sleeping state determined by the sleeping condition-determining means is read from the health information storing means, and the health information is presented to the user along with the determined sleeping state. In such a case, a scientifically established relationship between indicators of the sleeping state and the physical and mental conditions included in the health information corresponding to the sleeping state is stored in a health information storing unit. Indicators for the sleeping state used include sleeping hours, ease of getting to sleep, ease of waking, interruption of sleep, hours of nap, and changes in heartbeats during sleep. The physical and mental conditions included in the health information include a decrease in brain function, ability to control emotion, tendency to go out of control, lifestyle-related diseases (accelerated senescence, lifespan, obesity, and high blood pressure), skin roughness, dementia, and stress.

Regarding the predicted life expectancy of human, a technique that uses a computer is known (for example, refer to Japanese Laid-open Patent Publication No. 2004-021784). In calculating the predicted life expectancy, personal data related to the current physical information and data such as average life expectancy data are referred.

Another technique for calculating a predicted life expectancy value has been known in which basic data regarding healthy life expectancy prediction derived from the health check results are used (for example, refer to Japanese Laid-open Patent Publications No. 2003-167959 and No. 2007-287184). This technique requires input of the results of health check through a health check result input procedure. The predicted healthy life expectancy value is calculated for every individual person, and the calculation result is displayed in displaying means.

According to the conventional realistic life expectancy estimations, a highly accurate estimation of life expectancy has been attempted by utilizing various information of many aspects, such as human health information and daily life information. Such a life expectancy estimation is excessively thorough and information handled there is complicated, making it preferable to obtain relevant information. In other words, many pieces of information regarding vital signs indicative of biological conditions are relevant to the life expectancy estimation.

However, it is cumbersome to use such a wide range of information. Although these techniques may be practical for calculating realistic life expectancy, they are not suited for simple calculation of the virtual life expectancy. Calculation of life expectancy on the basis of the blood pressure information, pulse rates, cholesterol values, and the illness history of the user and user's family members written down on the medical records in hospitals, the results of health checks, etc., is highly specialized.

Among the vital signs, pulse information is useful information correlated to the life expectancy of a person. However, constantly monitoring the pulse information and counting pulses by carrying complicated pulse meters such as electrocardiographs or the like is hardly practical. Thus, for the health management purposes and the like, simple calculation of a virtual life expectancy and presentation of the calculated values are desired.

### SUMMARY

According to an aspect of the invention, a life expectancy estimation apparatus for estimating a predicted value of life expectancy by pulse information of a biological body includes an obtaining unit operable to obtain the pulse information; a storage for storing a record of pulse information of a biological body including a plurality of entries of pulse beat rates of the biological body over a period of time; and a calculation unit operable to calculate an estimated value of life expectancy from the number of remaining pulse beats and the pulse beat rates, the number of the remaining pulse beats being estimated by subtracting the number of total pulse beats in the past estimated from the record of the pulse information from the predetermined estimated value of total pulse beats in a whole life time of the biological body.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is a drawing illustrating the functional configuration of a life expectancy estimation apparatus;

FIG. 2 is a diagram illustrating the configuration of a data storage unit;

FIG. 3 is a diagram illustrating the state in which the pulse is measured with a camera-type pulse meter;

FIG. 4 is a flowchart illustrating a pulse detection process;

FIG. 5 is a flowchart illustrating a pulse rate calculation process;

FIG. 6 is a flowchart illustrating a life expectancy calculation process;

FIG. 7 is a diagram illustrating the history of the pulse detection process and the pulse detection timings;

FIG. 8 is a flowchart illustrating a process executed by a posting processor;

FIG. 9 is a diagram illustrating a posting summary table;

FIGS. 10A to 10C are diagrams illustrating examples of displays of evaluated values and advice;

FIGS. 11A to 11C are diagrams illustrating examples of displays of advice on the pulse measuring state;

FIG. 12 is a flowchart illustrating a process executed in a history managing unit according to a second embodiment;

FIG. 13 is a flowchart illustrating a process executed in a life expectancy calculating processor according to the second embodiment;

FIG. 14 is a diagram illustrating a hardware configuration of a portable terminal device according to a third embodiment;

FIG. 15 is a diagram illustrating the appearance of a cellular phone in an open state;

FIG. 16 is a diagram illustrating the appearance of the cellular phone in a closed state;

FIG. 17 is a diagram illustrating the structure of an ear clip-type pulse meter according to a fourth embodiment;

FIG. 18 is a drawing illustrating how to fasten the ear clip-type pulse meter;

FIG. 19 is a flowchart illustrating a pulse detection process;

FIGS. 20A to 20C are diagrams illustrating examples of displays of advice on the pulse measuring state;

FIG. 21 is a diagram illustrating a PDA according to another embodiment; and

FIG. 22 is a diagram illustrating a PC according to another embodiment.

### DESCRIPTION OF EMBODIMENTS

### First Embodiment

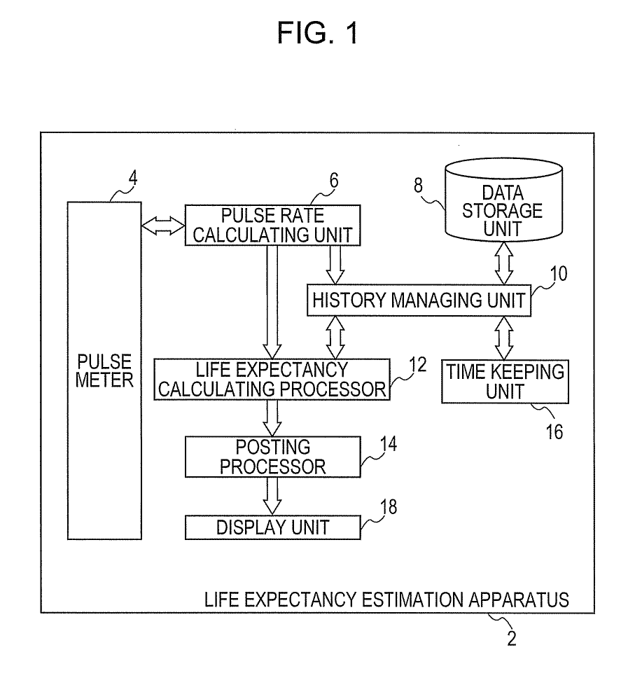

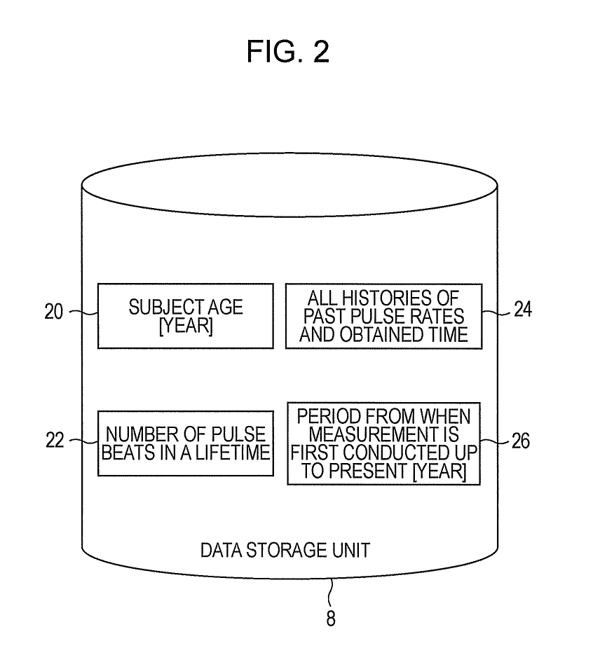

A first embodiment is described with reference to FIGS. 1 and 2. FIG. 1 is a drawing illustrating the functional configuration of a life expectancy estimation apparatus. FIG. 2 is a diagram illustrating the configuration of a data storage unit. The configurations illustrated in FIGS. 1 and 2 are merely examples and thus are not limiting.

For example, a life expectancy estimation apparatus 2 is one example of life expectancy estimation means that uses a computer as computing means. The life expectancy estimation apparatus 2 obtains pulse information of a user, uses the recorded past pulse information and age information, etc., and calculates virtual life expectancy information (e.g. predicted life expectancy value) on the basis of the assumption of the number of pulse beats in a lifetime (number of pulses in a lifetime). The calculated result may be presented to the user through the life expectancy estimation apparatus 2 or may be presented on a server via a network. The life expectancy estimation apparatus 2 may be designed as a portable device or may be constituted by or mounted in a portable terminal device or a personal computer. As illustrated in FIG. 1, the life expectancy estimation apparatus 2 includes a pulse meter 4, a pulse rate calculating unit 6, a data storage unit 8, a history managing unit 10, a life expectancy calculating processor 12, a posting processor 14, a time keeping unit 16, and a display unit 18.

The pulse meter 4 is one example of pulse information obtaining means for obtaining pulse information and detects pulses on the basis of the state of blood flow of a pulse detection target such as a finger or an earlobe. The detected pulse information is sent to the pulse rate calculating unit 6.

The pulse rate calculating unit 6 is an example of processing means for calculating the pulse rate or determining the detection state of the pulse and calculates the pulse rate on the basis of the pulse information received from the pulse meter 4. The pulse rate calculating unit 6 then sends the calculated pulse rate to the history managing unit 10 and the life expectancy calculating processor 12. In the pulse detection process carried out by the pulse meter 4, whether a finger is positioned appropriately or an earlobe is clipped appropriately is determined and the state of failing to place a detection target, i.e., a finger, on the pulse meter 4 is detected, and the results are posted to the posting processor 14.

The data storage unit 8 is an example of a pulse information recording unit for recording the detected pulse information, the calculated pulse rate, the age information of the user, etc. In the life expectancy calculating process, the stored pulse information and the like are read out and posted to the life expectancy calculating processor 12.

The history managing unit 10 is means for managing the obtained data by adding time information and the like. Once the pulse rate information is obtained from the pulse rate calculating unit 6, the history managing unit 10 correlates the obtained pulse rate information with the obtained time information and the age information of the subject and stores the information in the data storage unit 8.

The life expectancy calculating processor 12 is an example of a life expectancy computing unit for calculating the life expectancy value (predicted life expectancy value) by the equation described below on the basis of the calculated pulse rate. The life expectancy calculating processor 12 obtains the calculated pulse rate, the age information, the past pulse information, etc., from the data storage unit 8 through the history managing unit 10, and calculates the life expectancy by using the equation below on the basis of the obtained information.

The posting processor 14 is an example of posting means for posting the life expectancy value calculated by the life expectancy calculating processor 12 to the user, and outputs the calculation result of the life expectancy value and message information containing advice information for maintaining health set according to the calculation result, which are displayed in the display unit 18.

The time keeping unit 16 is means for keeping the timing of pulse measurement, the time and period at which the pulse information is obtained, etc., and for counting the number of times the pulse is measured as described below.

The display unit 18 displays the calculated life expectancy information, the advice information, and the like to the user of the life expectancy estimation apparatus 2. For example, the display unit 18 may display the calculated life expectancy value, and, in addition to or instead of the calculated predicted life expectancy value, a message such as measures and advice on the health condition that takes into account the age information of the user or the like.

As illustrated in FIG. 2, the data storage unit 8 stores, for example, information of the user of the life expectancy estimation apparatus 2, such as “subject age (year)” 20, a “number of pulse beats in lifetime” 22, “all histories of the pulse rate and obtained time in the past” 24, “the period (years) from when the measurement is first taken up to the present” 26, and the like. As for the “subject age” 20, the age information of the user at the present may be stored by carrying out an age-adding process by a calendar function or the like on the personal information such as age and the like registered when the subject started using the life expectancy estimation apparatus 2. Alternatively, the age may be input every time the life expectancy estimation apparatus 2 is used.

For the “number of pulse beats in lifetime” 22, published information such as statistic information in the medical field or the like is registered in advance. The predestinated number of pulse beats registered is 2 billion, for example. In the life expectancy calculating process described below, the number of years taken to reach 2 billion pulse beats is predicted and calculated on the basis of the information regarding the pulse rate up to the present. This 2 billion pulse beats is merely an example and not limiting. For example, the number of pulse beats may be modified in response to the updates of the published statistic information or any value chosen and input by the user.

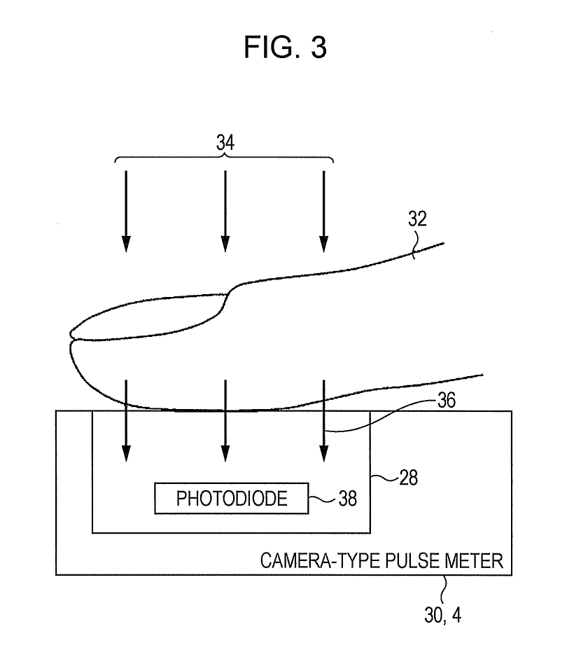

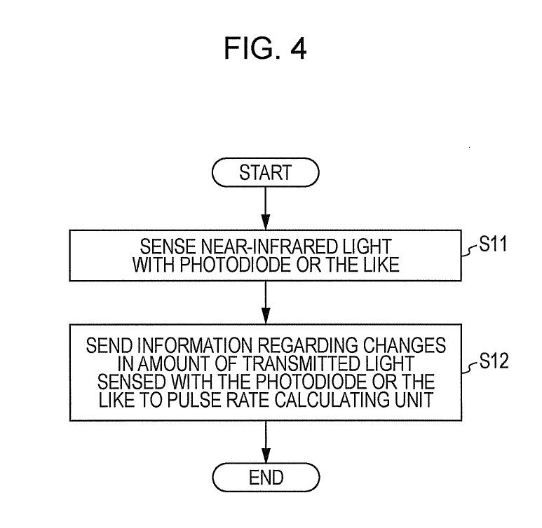

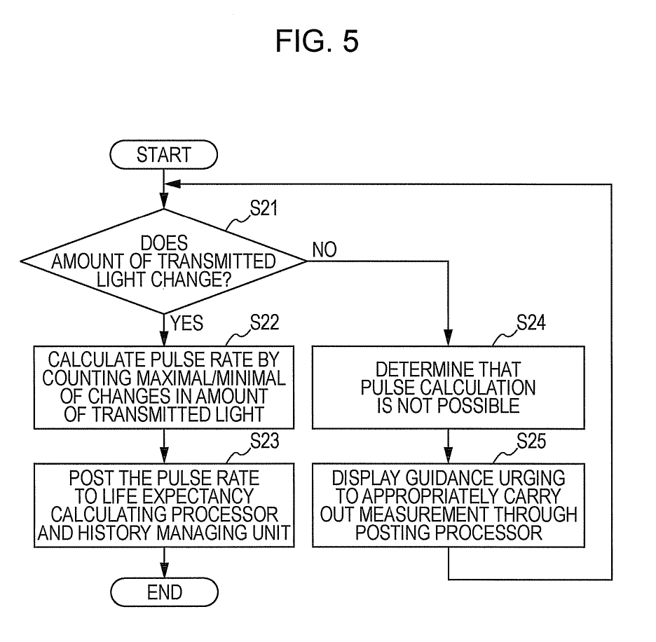

Next, the pulse detection by the pulse meter is described with reference to FIGS. 3, 4, and 5. FIG. 3 is a diagram illustrating the state in which the pulse is measured with a camera-type pulse meter; FIG. 4 is a flowchart illustrating a pulse detection process; and FIG. 5 is a flowchart illustrating a pulse rate calculation process. The structures and procedures and the like illustrated in FIGS. 3, 4, and 5 are merely examples and are not limiting.

One example of the pulse meter 4 is a camera-type pulse meter 30 equipped with a camera 28 for obtaining pulse information. In this camera-type pulse meter 30, a user places a pulse detection target, e.g., a finger 32, on the camera 28, and transmitted light 36, which is light in the near-infrared wavelength range that has transmitted through the finger 32, is captured relative to light 34, such as florescent light, sunlight, and the like, incident (applied) on the finger 32. Of the components of the incident light 34, the near-infrared rays have a characteristic of being absorbed by hemoglobin in the blood. At a timing when the blood flow is high, the amount of near-infrared transmitted light decreases, and at a timing when the blood flow is low, the amount of transmitted light increases. Since the blood flow increases and decreases in response to the pulses, pulses are detected by sensing the amount of transmitted light with a photodiode 38 in the camera 28 or on the basis of the changes in luminance components captured in camera images.

As illustrated in FIG. 4, the pulse detection process involving the camera-type pulse meter 30 includes sensing the near-infrared light (transmitted light 36) that has transmitted through the detection target, i.e. the finger 32, with the photodiode 38 or the like relative to the incident (applied) light 34 such as sunlight (step S11). Then the information related to changes in amounts of transmitted light 36 sensed is sent to the pulse rate calculating unit 6 (step S12).

The pulse rate calculating unit 6 that has obtained the information related to the changes performs the pulse calculation process on the basis of the information related to the changes, as illustrated in FIG. 5.

Then whether the amount of transmitted light changed or not is determined (step S21). In other words, if the amount of the transmitted light does not change, pulse detection is not accurately conducted. For example, it is possible that the finger 32 is not appropriately placed on the camera 28.

In this determination, when the amount of transmitted light is changed (YES in step S21), i.e., when the pulse can be detected, the maximal and the minimal of the changes in the amount of transmitted light are counted and the pulse rate is calculated (step S22). The calculated pulse rate is posted to the life expectancy calculating processor 12 and the history managing unit 10 (step S23).

When the changes in the amount of transmitted light is not detected (NO in step S21), it is determined that the pulse calculation is not possible (step S24). In other words, in the case of a conventional camera-type pulse meter 30, it is determined that the user is not appropriately placing the finger 32. Thus, this is posted to the posting processor 14, and a guidance urging the user to appropriately carry out measurement is provided from the posting processor 14 through a display unit 118 (step S25).

Such guidance posting related to how to place the measurement target may be done by audio through speakers and the like, for example, instead of the display through the display unit 18.

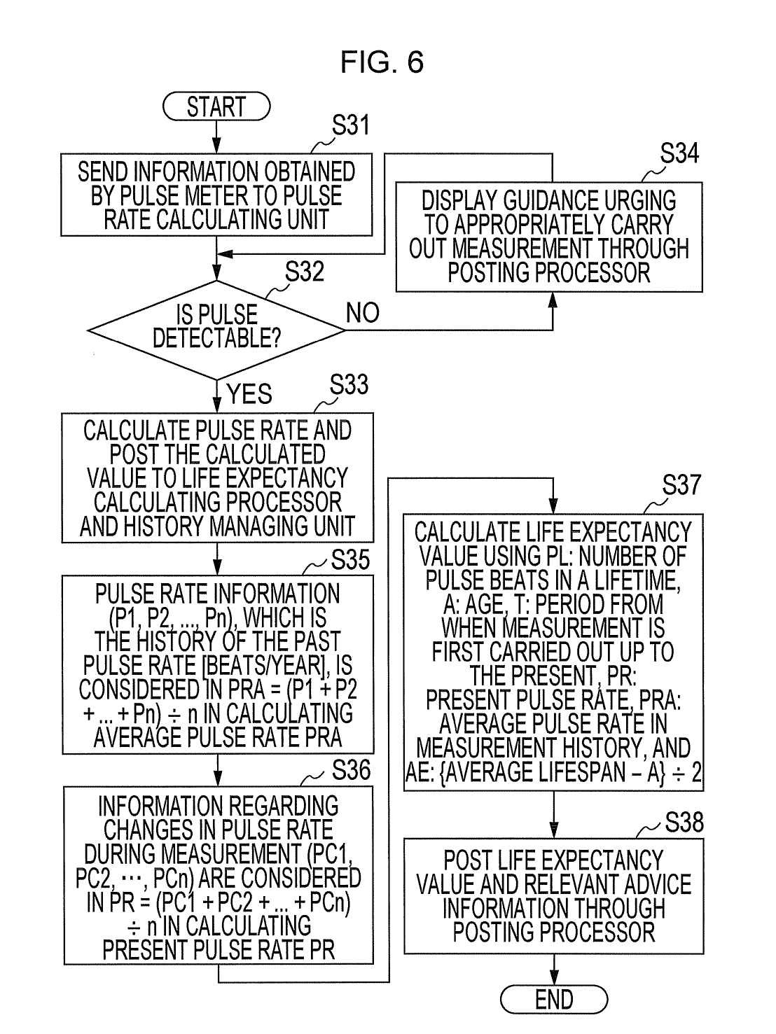

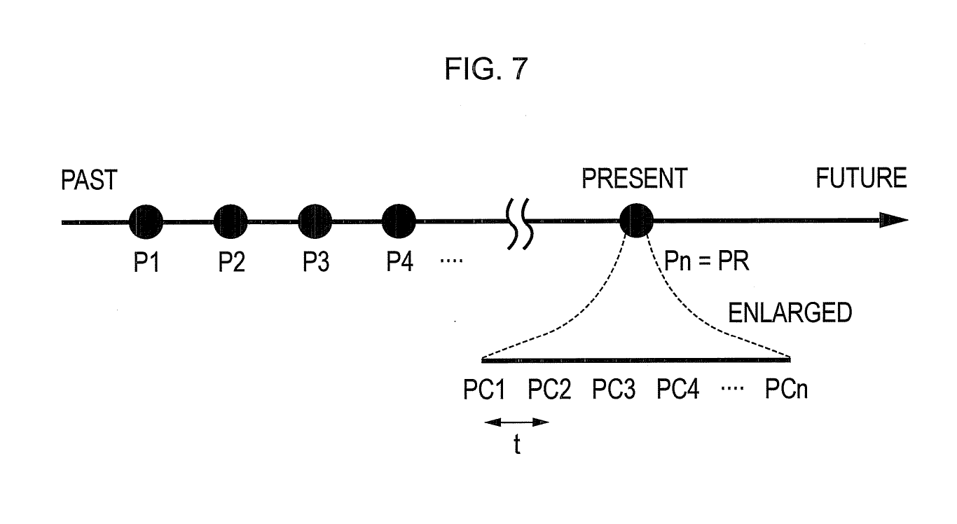

Next, the contents of processes of the life expectancy estimation method and the life expectancy estimation program are described with reference to FIGS. 6 and 7. FIG. 6 is a flowchart illustrating a life expectancy calculation process and FIG. 7 is a diagram illustrating the history of the pulse detection process and the pulse detection timings. The process contents and procedures illustrated in FIGS. 6 and 7 are merely examples and thus are not limiting.

In this life expectancy calculating process, the number of pulse beats of the user up to the present is calculated on the basis of the recorded past pulse information. Then the number of pulse beats up to the present is subtracted from the number of pulse beats in a lifetime to calculate the number of remaining pulse beats, and the number of remaining pulse beats and the pulse rate at the present are referred to calculate a predicted virtual life expectancy value. Then advice information related to the health care and the like is posted to the user on the basis of the predicted life expectancy value.

As the process for obtaining the present pulse information, the information obtained in the pulse meter 4 is sent to the pulse rate calculating unit 6 (step S31) as mentioned earlier. Then whether the pulse can be detected or not is determined in the pulse rate calculating unit 6 (step S32).

When it is determined that the pulse can be detected (YES in step S32), the pulse rate is calculated in the pulse rate calculating unit 6, and the calculated result and information regarding changes in pulse rate and the like are sent to the life expectancy calculating processor 12 and the history managing unit 10 (step S33).

When it is determined that the pulse is not detected (NO in step S32), the posting processor 14 outputs and displays a guidance urging the use to carry out pulse detection measurement appropriately through the display unit 18 (step S34), and the detection process of step S32 is continued again.

Next, the average pulse rate PRA (beats/year) in the history of the past pulse rates is calculated (step S35). The average pulse rate PRA is, as illustrated in FIG. 7, calculated by using calculated pulse rate information P1, P2, P3, . . . , Pn from past to present. Thus, the data storage unit 8 and the history managing unit 10 of the life expectancy estimation apparatus 2 store the pulse rate information up to the present obtained by running the life expectancy calculation process or the like, the time at which the information is obtained, and the like.

The pulse rate information stored is not limited to the pulse rate information obtained by executing the life expectancy calculation process. For example, pulse information or pulse rate information obtained by executing other programs may be utilized.

The process then proceeds to a pulse rate calculating process that calculates the pulse rate from past to present. The pulse rate information P1, P2, . . . , Pn stored in the data storage unit 8 are read on the basis of the measurement history of the history managing unit 10, and the average pulse rate PRA of the past is calculated by the equation below:

PRA=(P1+P2+ . . . +Pn)/n (1)

As described above, since the past pulse rate information can be utilized in calculating the life expectancy, the average pulse rate of the user can be considered in calculating the pulse rate up to the present.

Information regarding changes in pulse rate during measurement is used in calculating the present pulse rate PR (step S36). That is, as illustrated in FIG. 7, in calculating the present pulse rate PR, pulse rate change information PC1, PC2, . . . , PCn obtained through a plurality of times of measurement is used. The equation for calculating the present pulse rate PR is illustrated below in which an average is taken from pulse rate change information obtained every predetermined time t, e.g., one second, for a number n of times of measurement. The number n and the predetermined time are counted by the time keeping unit 16.

PR=(PC1+PC2+ . . . . +PCn)/n (2)

The pulse rate calculated every predetermined time somewhat changes even when the user is at rest. However, since the average of the results of measurement conducted a plurality of times is used, the life expectancy can be calculated while considering the changes in pulse rate during measurement.

Next, the life expectancy value is calculated (step S37). The equation for calculating the life expectancy value is illustrated below.

Life Expectancy Value={PL−PRA*T−Σ(60×24×365×(A−T−n))}÷(PR−60×24×365×AE) (3)

(Σ includes from n=0 to n=(A−T))

In Equation (3), A represents age (years) and T represents the period (years) from the time measurement is first conducted to the present, i.e., the period of acquiring the pulse information. The life expectancy calculating processor 12 uses the pulse rate, heartbeat changes, age information, etc., obtained from the history managing unit 10 and takes into account the number of pulse beats in a lifetime, the measurement period, etc. In such a case, the values calculated by Equation (1) of step S35 and Equation (2) of step S36 are used as the average pulse rate PRA and the present pulse rate PR. PL represents the number of pulses (beats) in a lifetime (e.g. 2 billion beats). In Equation (3), (PRA*T) calculates the number of past pulse beats by multiplying the average pulse rate PRA and the measurement period T (years). AE represents a value that considers future changes in pulse and is calculated by the equation below.

In Equation (3), as described above, the number of remaining pulse beats is determined by subtracting the number of pulse beats to the present from the number of pulse beats in a lifetime, and the predicted life expectancy value is calculated from the value AE that considers the current pulse rate PR and the future pulse changes.

AE={Average Lifespan (e.g., 80 [years])−A}÷2 (4)

Note that the average lifespan described above may be the number of years that is publicly available or may be any number input by the user, for example. Alternatively, the latest data regarding the average lifespan at the time of calculating the life expectancy may be downloaded by using a communication function or the like of the portable terminal device. The period T in the equation described above may be counted by the time keeping unit 16 or may be freely input by the user.

The result of life expectancy calculation is sent to the posting processor 14. The life expectancy value calculated is posted to the user along with advice information regarding the health condition corresponding to the life expectancy value and the like (step S38). The contents of the advice information regarding the health condition and the like change depending on the calculated life expectancy value. A table 50 (FIG. 9) illustrating the correlation between the life expectancy value and the advice information may be preliminarily stored in the data storage unit 8 and the data corresponding to the calculated value may be read by the command from the posting processor 14 and posted to the user.

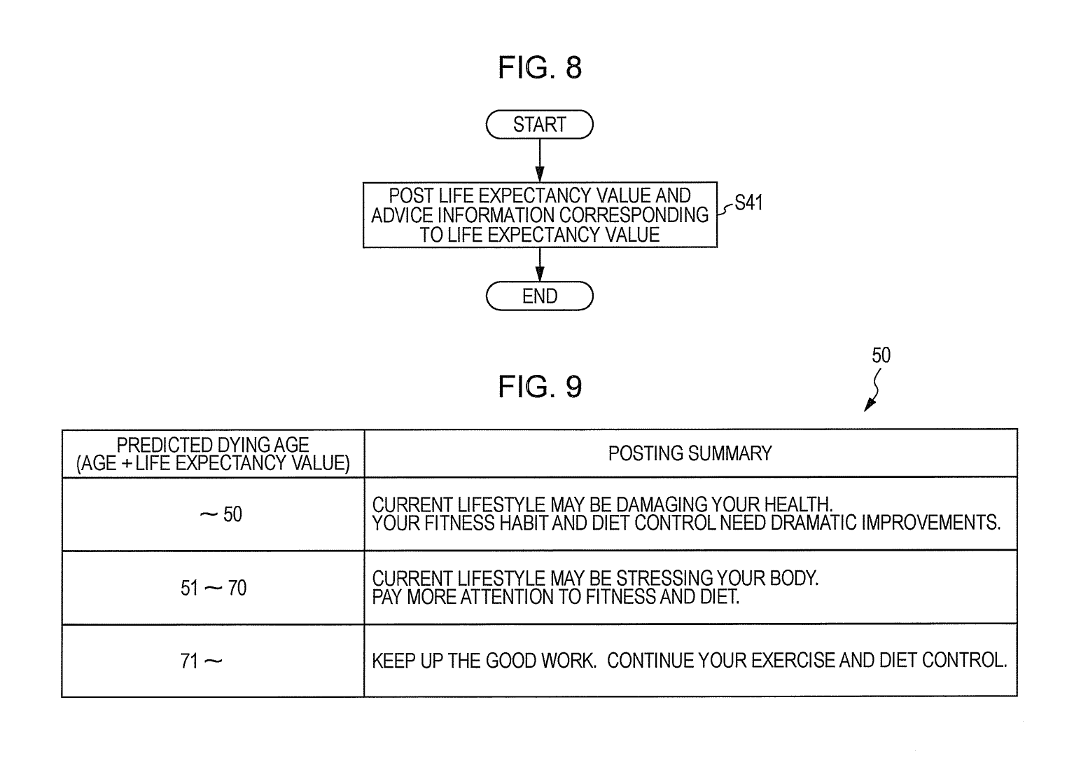

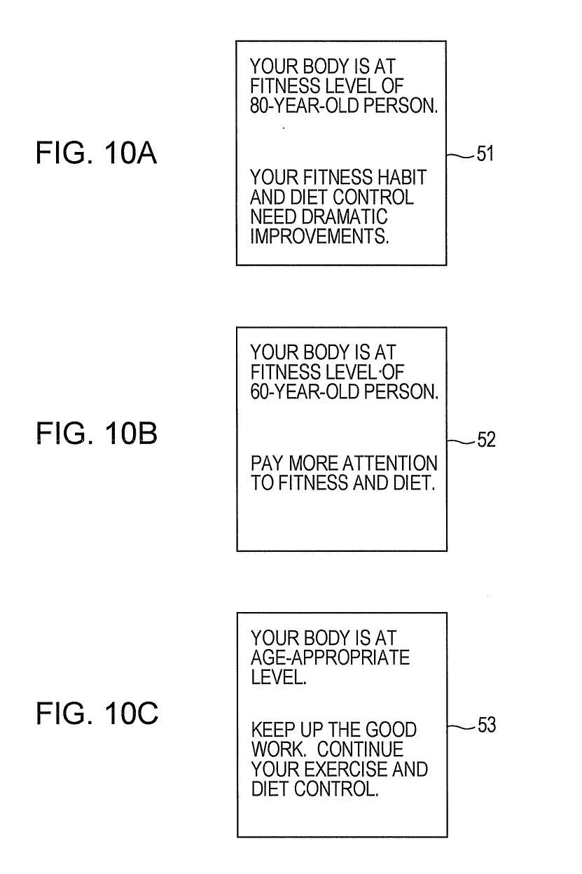

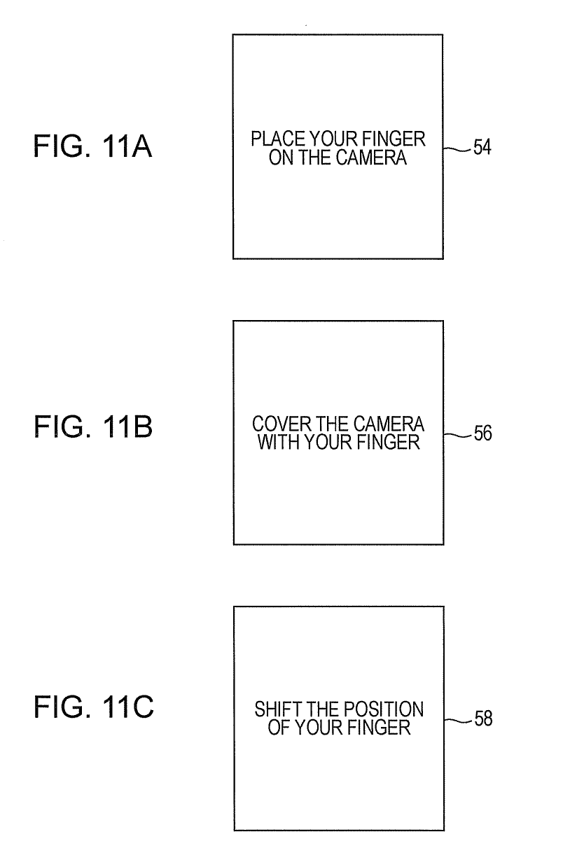

The posting process of the life expectancy estimation apparatus will now be described with reference to FIGS. 8, 9, 10, and 11. FIG. 8 is a flowchart illustrating the process executed by the posting processor. FIG. 9 illustrates a posting summary table. FIGS. 10A to 10C include diagrams illustrating examples of displays of evaluated values and advice. FIGS. 11A to 11C include diagrams illustrating examples of displays of advice on the pulse measuring state. The contents illustrated in FIGS. 8, 9, 10, and 11 are merely examples and are not limiting.

The posting processor 14 that has received the calculated results from the life expectancy calculating processor 12 posts the life expectancy value and advice information corresponding to the life expectancy value (step S41). In the life expectancy estimation apparatus 2, not only the calculated life expectancy value but also the advice corresponding to the life expectancy value is displayed to improve the health consciousness of the user.

For displaying the advice information by the posting processor 14, for example, as illustrated in FIG. 9, a posting summary table 50 is set so that the summary of the posting changes on the basis of a particular standard. In the posting summary table 50, the contents of the advice display are changed on the basis of the predicted dying age obtained by adding the calculated life expectancy and the age information. To be more specific, when the predicted dying age is 50 or less, a posting summary that reads “Current lifestyle may be damaging your health. Your fitness habit and diet control need dramatic improvements” may be displayed. When the predicted dying age is 51 to 70, a posting summary that reads “Current lifestyle may be stressing your body. Pay more attention to fitness and diet” may be displayed. When the predicted dying age is 71 or over, the posting summary that reads “Keep up the good work. Continue your exercise and diet control” may be displayed.

As a result, the posting processor 14 determines the range of the posting summary table 50 the calculated predicted dying age belongs and posts the set advice information to the user. The posting of the advice information may be done through display in the display unit 18, by sound or voice, or the like.

The predicted dying age set in the posting summary table 50 is not limited to a fixed value. For example, the value may be changed depending on the current age of the user input.

Examples of the display of advice information according to the criteria of the posting summary table 50 will now be described. As illustrated in FIGS. 10A to 10C, the display unit 18 displays the above-mentioned advice, the evaluation value evaluating the measured present health condition of the user by age, and the like. As for the examples of the advice display, when the predicted dying age is 50 or less, as illustrated in FIG. 10A, a display 51 that reads “Your body is at a fitness level of 80-year-old person. Your fitness habit and diet control need dramatic improvements” may be presented. When the predicted dying age is 51 to 70, as illustrated in FIG. 10B, a display 52 that reads “Your body is at a fitness level of 60-year-old person. Pay more attention to fitness and diet” may be presented. When the predicted dying age is 71 or over, as illustrated in FIG. 10C, a display 53 that reads “Your body is at age-appropriate level. Keep up the good work. Continue your exercise and diet control” may be presented.

As for the advice displays 51 to 53 evaluating the health condition, for example, evaluation may be conducted on the basis of a table or the like that can preliminarily evaluate the health condition from the calculated life expectancy value. Alternatively, calculation may be conducted by using the present age information and the average lifespan or the like.

The posting processor 14 and the display unit 18 display advice urging the user to appropriately conduct measurement in the pulse measuring process (step S25: FIG. 5, step S34: FIG. 6). For example, when the life expectancy estimating function is started, as illustrated in FIG. 11A, a display 54 urging the user to place the finger 32 on the camera 28 is presented. For example, when the finger 32 is not placed on the camera 28 or the finger 32 is not appropriately detected, a display 56 urging the user to cover the camera 28 with the finger 32 is presented, as illustrated in FIG. 11B. For example, when the finger 32 is detected but the pulse is not detected, a display 58 urging the user to shift the position of the finger 32 is presented, as illustrated in FIG. 11C.

In this manner, changes in pulse rate during measurement and the like can be taken into consideration and thus a more practical life expectancy value virtually predicted on the basis of the present health condition can be calculated. Moreover, a portable terminal device can be used to calculate the life expectancy value. The calculated life expectancy value can be displayed along with the advice information corresponding to the life expectancy value to improve the health consciousness of the user. Furthermore, in acquiring the pulse information, measurement advice such as how to place a finger and the like are displayed so that accurate pulse information can be acquired and a more practical life expectancy value can be calculated.

The features and advantages of the embodiment described thus far are as follows.

(1) In the case where the average (standard) pulse rate for each age and sex is investigated/published and the value of the average lifespan is published, the life expectancy value can be calculated highly accurately by calculating the average number of pulse beats in a lifetime.

(2) Future changes in pulse rate are modeled as “{Published Average Lifespan (e.g., 80 years)−A}÷2” and the history of past pulse rates and changes in pulse rate during measurement are taken in consideration so that highly accurate pulse information is obtained. By using such pulse information, a practical life expectancy value can be calculated at higher calculation accuracy.

(3) The calculated life expectancy information can be presented to the user. Presentation of such information provides the user with an opportunity to take better care of oneself. A timely life expectancy information can be obtained on the basis of the past history and present measured values and thus useful and effective life expectancy information can be provided.

### Second Embodiment

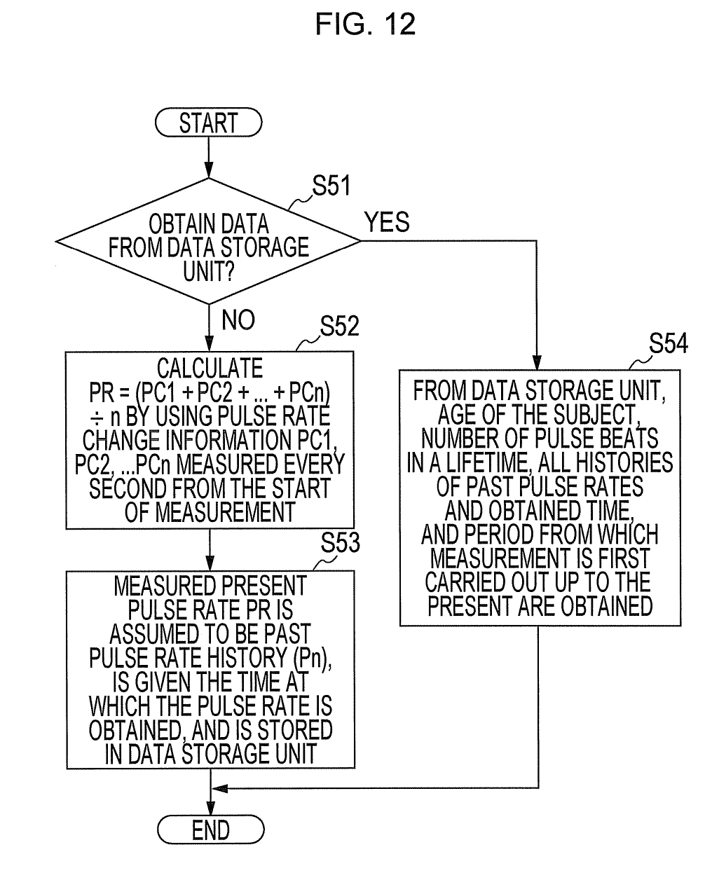

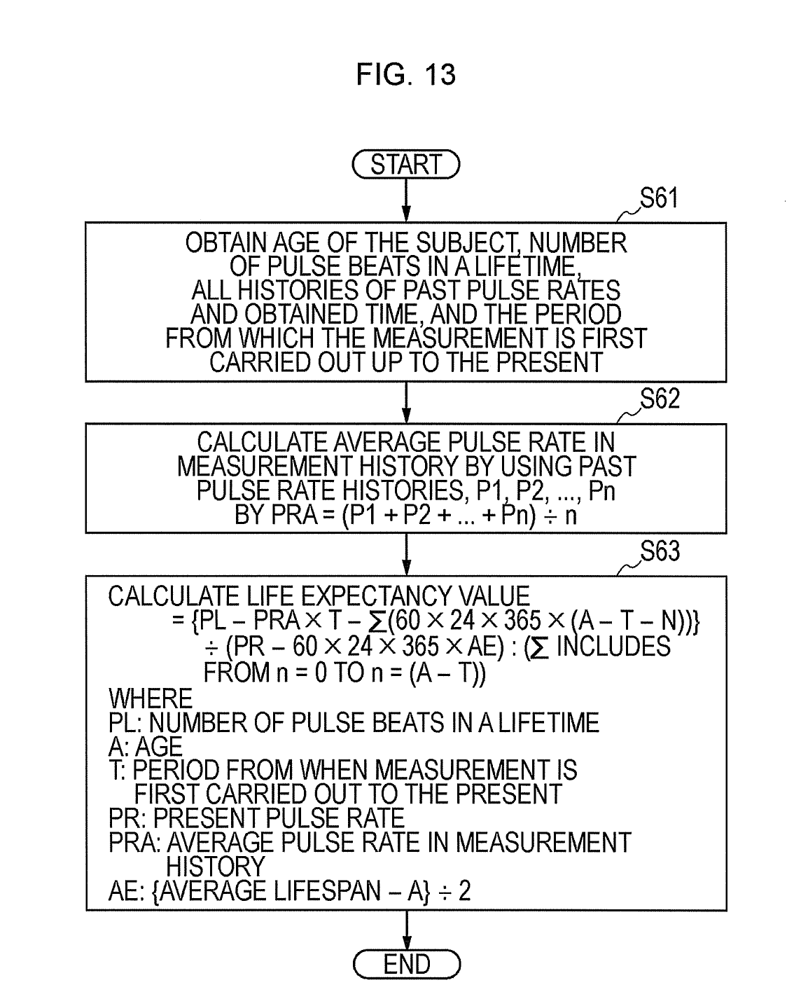

Next, a second embodiment is described with reference to FIGS. 12 and 13. FIG. 12 is a flowchart illustrating a process executed in a history managing unit according to the second embodiment. FIG. 13 is a flowchart illustrating a process executed in a life expectancy calculating processor according to the second embodiment. The contents and procedures of the processes illustrated in FIGS. 12 and 13 are merely examples and are not limiting.

In this embodiment, the processes executed in the history managing unit 10 and the life expectancy calculating processor 12 of the life expectancy calculating apparatus 2 are specifically described.

When the life expectancy calculating process is started, the history managing unit 10 of the life expectancy calculating apparatus 2 receives the pulse rate change information from the pulse rate calculating unit 6 and uses the change information to calculate the present pulse rate PR used for calculating the life expectancy as described above. Upon a request from the life expectancy calculating processor 12, the history managing unit 10 reads the data used for life expectancy calculation from the data storage unit 8, and sends the data.

The history managing unit 10 determines whether to calculate the present pulse rate PR or obtain data from the data storage unit 8 (step S51). If it determines not to obtain the data (NO in step S51), the history managing unit 10 calculates the present pulse rate PR by Equation (2) above using the pulse rate change information PC1, PC2, . . . , PCn measured every second from start of the measurement (step S52). The calculated present pulse rate PR is assumed to be the past pulse rate history Pn, given the time at which the rate is obtained, and stored in the data storage unit 8 (step S53).

If the data is to be obtained (YES in step S51), the history managing unit 10 obtains data regarding the subject's age, the number of pulse beats in a lifetime, all histories of the past pulse rate and obtained time, and the period from when the measurement is first carried out up to the present from the data storage unit 8 (step S54), and sends the data to the life expectancy calculating processor 12 and the like.

As illustrated in FIG. 13, in order to execute life expectancy calculation, the life expectancy calculating processor 12 commands the history managing unit 10 to read out data regarding the subject's age, the number of pulse beats in a lifetime, all histories of the past pulse rate and obtained time, and the period from when the measurement is first carried out up to the present stored in the data storage unit 8 and obtains the data (step S61).

The average pulse rate PRA is calculated by Equation (1) above on the basis of the past pulse rate histories, P1, P2, . . . , Pn (step S62), and the life expectancy is calculated by Equation (3) above (step S63).

According to this configuration, with regard to the past calculation data stored in the data storage unit 8, since the history managing unit 10 counts the number of times of measurement conducted and calculates the data to be stored, or adds the obtained time information, the measurement history of the data up to the present becomes clear. Thus, a more practical life expectancy value can be calculated. The second embodiment also achieves the same advantages as the first embodiment.

### Third Embodiment

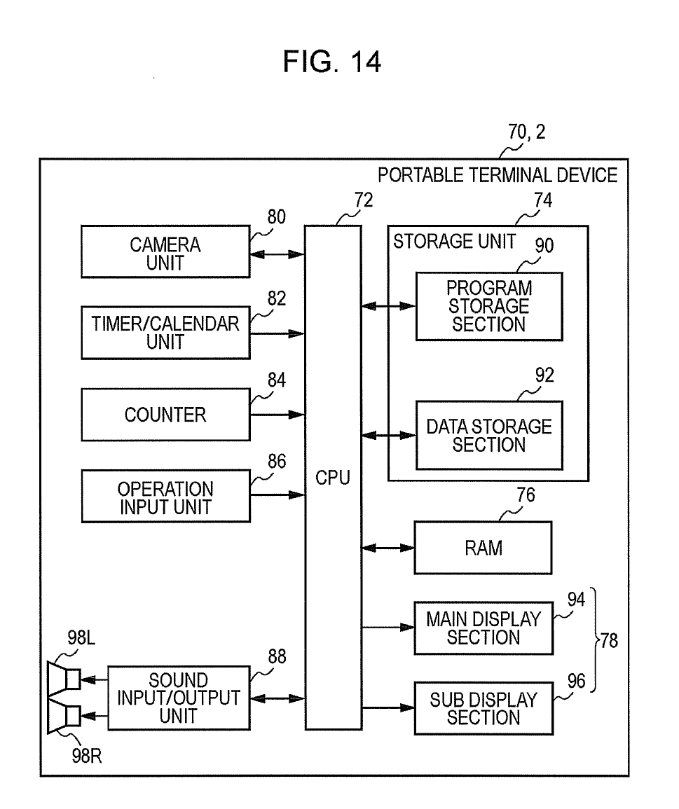

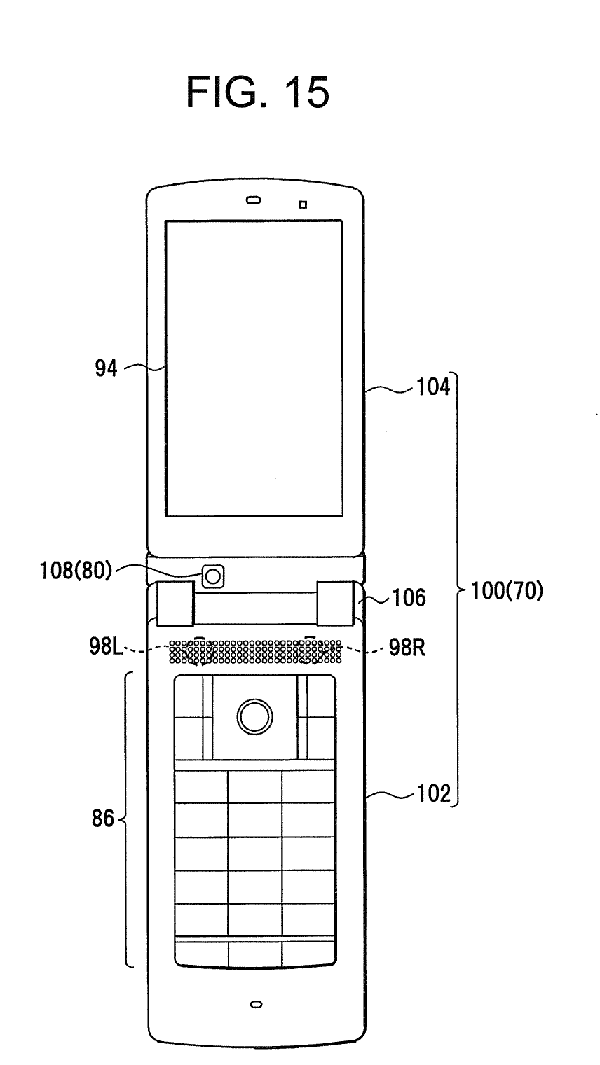

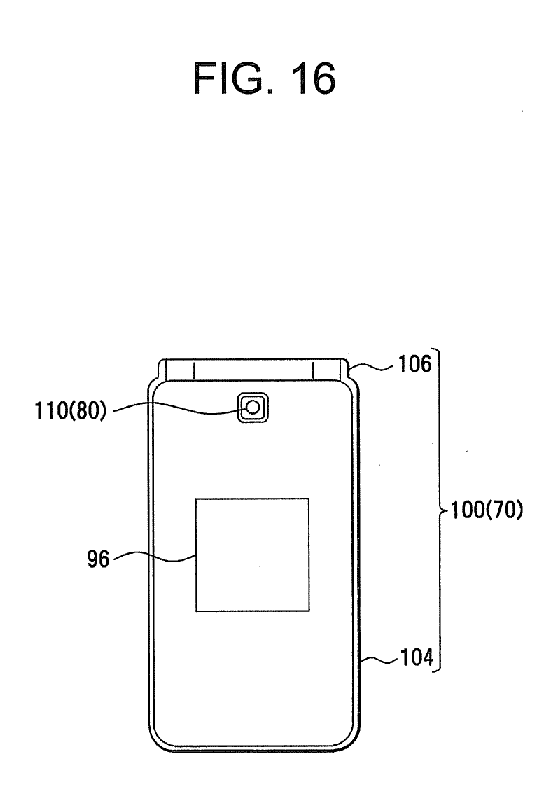

A configuration of a portable terminal device including a life expectancy calculating apparatus and having a life expectancy estimating function will now be described with reference to FIGS. 14, 15, and 16. FIG. 14 is a diagram illustrating a hardware configuration of a portable terminal device according to a third embodiment. FIG. 15 is a diagram illustrating the appearance of a cellular phone in an open state. FIG. 16 is a diagram illustrating the appearance of the cellular phone in a closed state. The configurations illustrated in FIGS. 14, 15, and 16 are merely examples and are not limiting.

A portable terminal device 70 includes, for example, a central processing unit (CPU) 72, a memory unit 74, a random-access memory (RAM) 76, a display unit 78, a camera unit 80, a timer/calendar unit 82, a counter 84, an operation input unit 86, a sound I/O unit 88, etc.

The CPU 72 is one example of computing means such as a program or the like performed in the portable terminal device 70 and runs the operating system (OS), the life expectancy calculating program, other application programs, and the like.

The memory unit 74 includes, for example, a program storage section 90 constituted by a read only memory (ROM) that stores the operation system of the portable terminal device 70, the life expectancy calculating program, and other programs and the like, and a data memory section 92 that constitutes the data storage unit 8, which is the pulse information recording unit for recording the pulse information described above.

The RAM 76 is a work area for executing the computation process and the like described above. The pulse rate calculating unit 6, the history managing unit 10, and the life expectancy calculating processor 12 of the life expectancy calculating apparatus 2 are configured by executing the life expectancy calculating program stored in the memory unit 74.

The display unit 78 constitutes the posting processor 14 serving as the information display means for displaying the calculated life expectancy information, the health advice, and the like described below. For example, the display unit 78 includes a main display section 94 and a sub display section 96 formed by liquid crystal displays (LCDs). The camera unit 80 is one example of image capturing means for capturing the image of a fingertip, palm, or the like, and is constituted by a digital camera or the like to make up the pulse meter 4 for reading the state of blood flow of the fingertip and the like.

The timer/calendar unit 82 constitutes the time keeping unit 16 of the life expectancy calculating apparatus 2 and is means for obtaining the time information, date information, etc. In the life expectancy calculating process, the timer/calendar unit 82 adds to the pulse information the time and date when the pulse information is obtained. The timer/calendar unit 82 also counts the measurement timing for the pulse meter 4 and obtains time information related to the period in which the pulse information is obtained, such as the time at which the life expectancy calculation function is started and the period elapsed from the time calculation is started. The counter 84 constitutes the time keeping unit 16 of the life expectancy estimation apparatus 2 and is means for counting the number of times the pulse measurement and the life expectancy calculation are performed.

The operation input unit 86 is information inputting means and has a keypad section including input keys for characters such as alphabets, cursor-control keys for selecting display information, and enter keys for determining the selected information. The sound I/O unit 88 includes, for example, speakers 98R and 98L and reproduces sound from a sound signal.

An example of the life expectancy estimation apparatus or the portable terminal device 70 incorporating the life expectancy estimation apparatus is a cellular phone 100 illustrated in FIG. 15. As for the appearance of the cellular phone 100 in an open state, the casing includes a lower casing 102 and an upper casing 104. The lower casing 102 and the upper casing 104 are connected by a hinge 106 to enable flipping. The operation input unit 86 and the speakers 98L and 98R are mounted in the lower casing 102. The operation input unit 86 includes the keypad section and the like. The main display section 94 and an in-camera 108 (80) are mounted in the upper casing 104.

As for the appearance of the cellular phone 100 in a closed state, as illustrated in FIG. 16, the sub display section 96 is disposed at the center of the outer side of the upper casing 104, and an out camera 110 (80) is mounted at the hinge 106 side.

If the portable terminal device 70 such as the cellular phone 100 has the life expectancy calculation function or incorporates the life expectancy estimation apparatus, the virtual life expectancy value can be easily presented through the portable terminal device 70 which is taken along with and used by the user in every day life. Thus, the same advantages as those of the first embodiment, such as presentation of the useful life expectancy information that provides an opportunity for improving the health, can be obtained.

### Fourth Embodiment

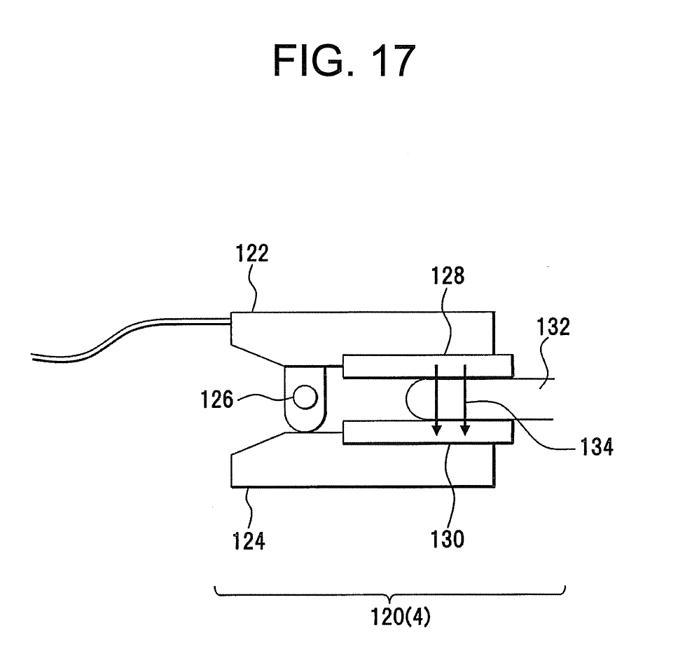

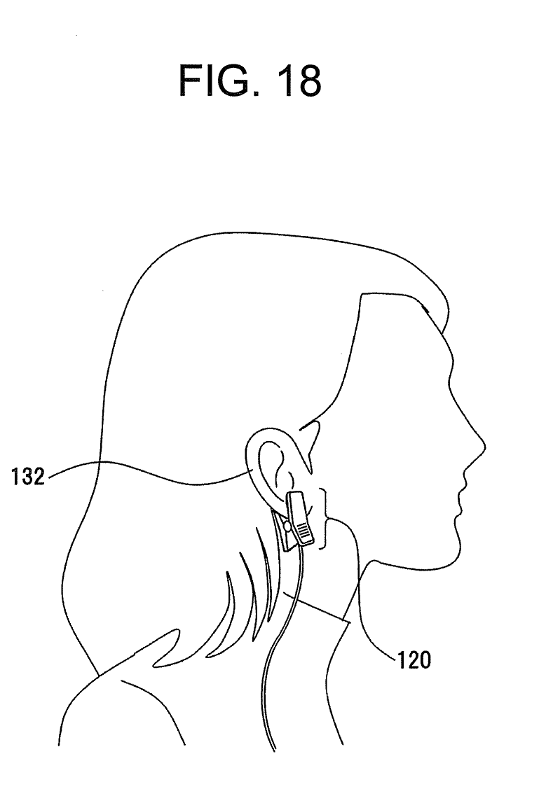

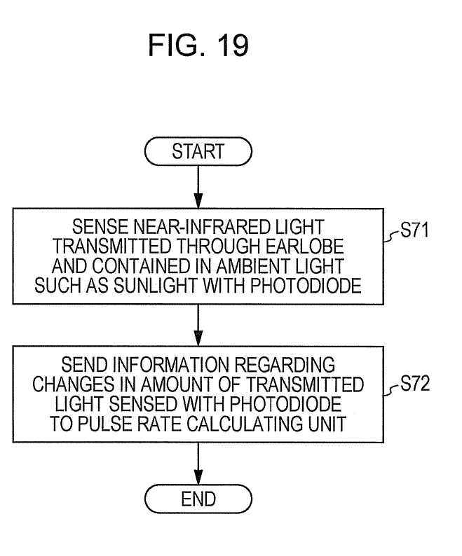

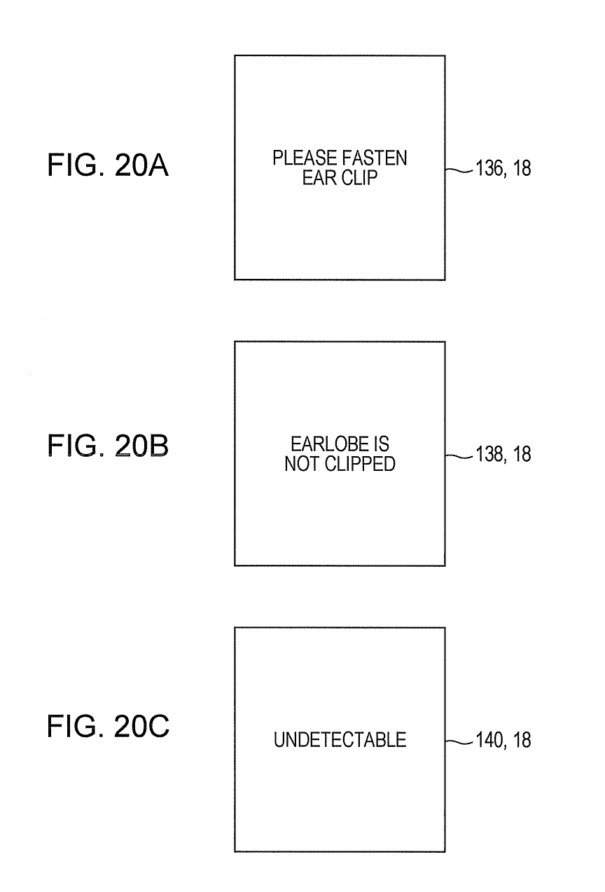

A fourth embodiment will now be described with reference to FIGS. 17, 18, 19, and 20. FIG. 17 is a diagram illustrating the structure of an ear clip-type pulse meter according to the fourth embodiment. FIG. 18 is a drawing illustrating how to fasten the ear clip-type pulse meter. FIG. 19 is a flowchart illustrating a pulse detection process. FIGS. 20A to 20C include diagrams illustrating examples of displays of advice on the pulse measuring state. The structure, the process content, and the process procedures illustrated in FIGS. 17, 18, 19, and 20 are merely examples and are not limiting. The description of the components that are similar to those described above is omitted.

In this embodiment, the structure of an ear clip-type pulse meter 120 and the measurement process therefor are described.

The ear clip-type pulse meter 120 is one example of the pulse meter 4. As illustrated in FIG. 17, the clip is constituted by two casings 122 and 124 connected to each other with a hinge 126. A near-infrared LED 128 and a photodiode 130 are respectively provided on surfaces of the casings 122 and 124 facing each other. As illustrated in FIG. 18, the casing 122 equipped with the near-infrared LED 128 and the casing 124 equipped with the photodiode 130 are arranged to clip an earlobe 132, which is the detection target of the user.

As for the measurement with the ear clip-type pulse meter 120, a near-infrared ray emitted from the near-infrared LED 128 is transmitted through the earlobe 132, and a transmitted light 134 is sensed with the photodiode 130. As a result, as in the pulse detection using the camera 28 described above, the pulse can be detected by the changes in amount of bloodstream on the basis of the amount of transmitted light.

As illustrated in FIG. 19, the pulse detection process by the ear clip-type pulse meter 120 involves sensing the near-infrared light transmitted through the earlobe 132 (transmitted light 134) with the photodiode 130 relative to the incident light emitted from the near-infrared LED 128 (step S71). Then the information regarding changes in amounts of the transmitted light 134 is sent to the pulse rate calculating unit 6 (step S72). The pulse rate calculating unit 6 executes the pulse calculating process on the basis of the change information.

Regarding the pulse measuring condition, an advice display is presented through the display unit 18. The examples of displays are as follows, for example. When the life expectancy calculation function is started, a display 136 indicating “Please fasten ear clip” illustrated in FIG. 20A is presented. When the earlobe 132 is not clipped or the bloodstream of the earlobe is not appropriately detected, a display 138 indicating “Earlobe is not clipped” illustrated in FIG. 20B is presented. In addition, for example, when clipping of the earlobe 132 is detected but the pulse is not detected, a display 140 indicating “Undetectable” illustrated in FIG. 20C is presented.

The clip-type pulse detection is not limited to using the bloodstream of the earlobe. Alternatively, for example, the pulse may be detected by clipping a fingertip.

In this manner, the pulse can be detected and the life expectancy value can be calculated with a simple configuration. Since changes in pulse rates measured and the like are considered, more accurate pulse information can be acquired, and a practical life expectancy value can be calculated. Since an advice display indicating the pulse detection condition is presented, more accurate pulse information can be obtained and a practical life expectancy value can be calculated. Thus, this embodiment also achieves the same advantages as the embodiments described above.

### Other Embodiments

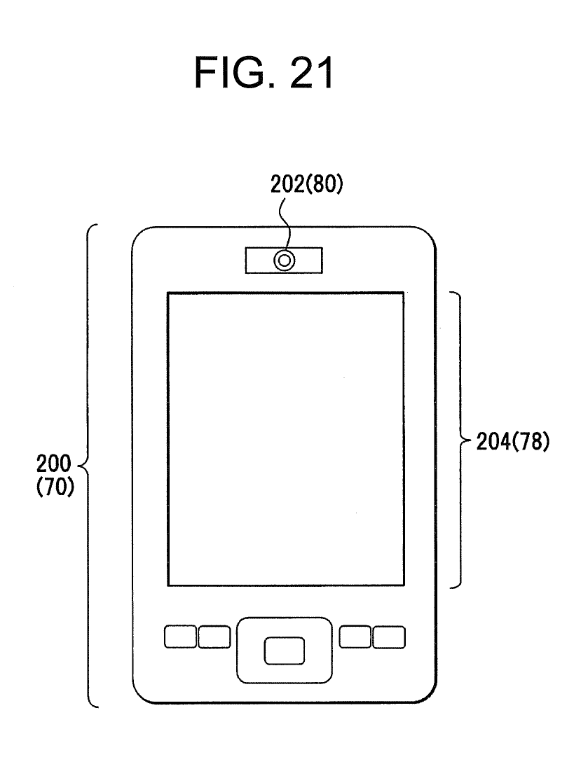

(1) In the embodiment described above, the cellular phone 100 (FIGS. 15 and 16) is given as an example of an electronic apparatus incorporating the life expectancy estimation apparatus and having the life expectancy estimating function. However, the present invention is not limited to the cellular phone 100. As illustrated in FIG. 21, the present invention may be applied to a personal digital assistant (PDA) 200, which is the portable terminal device 70 equipped with a pulse measuring function. The PDA 200 may include a pulse meter 4 constituted by a camera 202 (80) and a display unit 204. In addition, the ear clip-type pulse meter 120 or the like may be installed to the PDA 200.

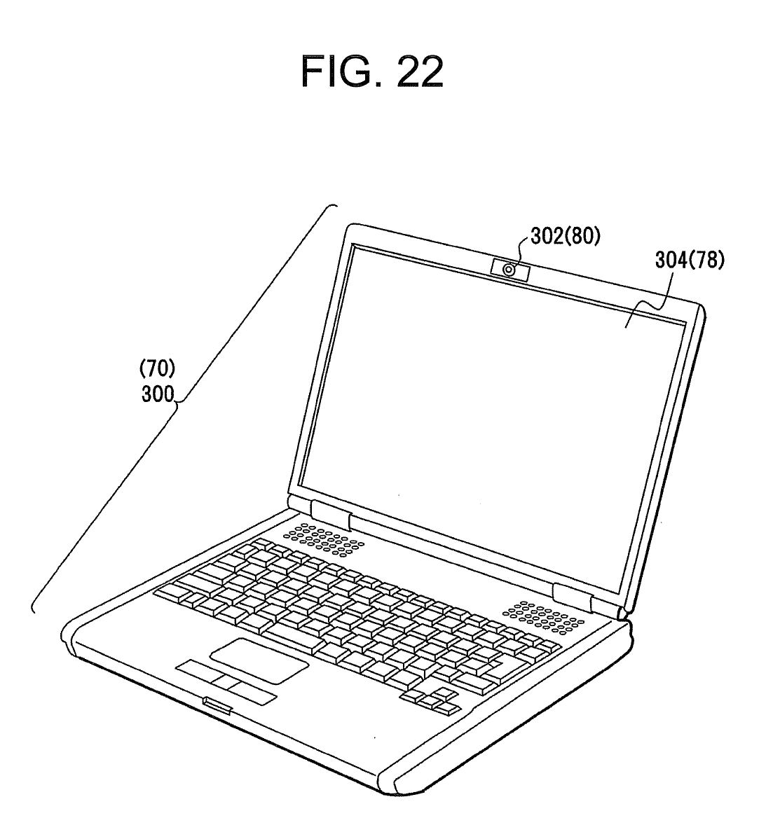

(2) Alternatively, as illustrated in FIG. 22, the life expectancy estimation apparatus 2 may be installed in a personal computer (PC) 300 having a pulse measuring function. The PC 300 includes a camera 302 and a display unit 304 to achieve the pulse measuring function. Other pulse measuring means may also be provided. The object can be achieved by this configuration also.

(3) For pulse detection in the embodiment described above, the flow of the blood in the fingertip or earlobe is detected on the basis of the amount of transmitted light, but the detection method is not limited to this. For example, the pulse may be detected by applying pressure on a wrist or the like or by measuring the sound of heartbeat of the actual user with a sound sensor or the like.

(4) In the embodiment described above, Equation (3) is given as one example of an equation for calculating the life expectancy value, but the equation is not limited to this. The life expectancy value can be calculated by using the following equation:

Life Expectancy Value={PL−PRA×T−Σ(60×24×365×(A−T−n))}÷(PR×60×24×365×AE) (5)

(Σ includes from n=0 to n=(A−T))

The life expectancy calculating processor 12 can perform computation on Equation (5) to calculate the life expectancy value.

The life expectancy value can be calculated by using the following equation:

Life Expectancy Value={PL−PRA×T−(60×24×365)×Σ((PRA)÷(60×24×365)−(A−T−n))}÷PR (6)

(Σ includes from n=0 to n=(A−T))

The life expectancy calculating processor 12 can perform computation on Equation (6) to calculate the life expectancy value. Equation (6) is derived from the following equation:

PL=(Life Expectancy Value)×PR+PRA×T+(60×24×365)×Σ((PRA)+(60×24×365)−(A−T−n)) (7)

In Equation (7), (Life Expectancy Value)×PR represents the number of future pulse beats, PRA×T represents the number of pulse beats from the time measurement is first conducted to the present and (60×24×365)×Σ((PRA)÷(60×24×365)−(A−T−n)) represents the number of pulse beats before acquiring the pulse information.

(5) In the embodiment described above, in Equations (3), (5) and (6) of the life expectancy estimation, both the age of the user and the period T from when the measurement is first carried out up to the present are used. However, this is not limiting, and, for example, the life expectancy estimation may be calculated by using only one of the age information and the period information. The object can also be achieved by such an arrangement.

(6) In the embodiment described above, the virtual life expectancy value is calculated and the calculated result is presented to the user through the display unit 18 on the life expectancy estimation apparatus 2. However, the present invention is not limited to such an arrangement. That is, when a portable device having a communication function such as the portable terminal device 70 or a personal computer (PC) are used, their information presentation functions such as the communication function and the printing function can be utilized. When the communication function is used, the computation results may be transferred to, presented in, and accumulated in a server device or another portable terminal device via a network. In such a case, the data storage unit 8 may be constituted by an external storage device, a computer, or the like so that the history information can be acquired as needed.

The life expectancy estimation apparatus, the life expectancy estimation method, and the life expectancy estimation program invention disclosed herein can achieve following advantages.

(1) A virtual life expectancy can be easily calculated by using the pulse information such as the number of pulse beats in a lifetime and measured pulse rates.

(2) A virtual life expectancy can be easily calculated using a portable terminal device. Since the predicted life expectancy value can be easily calculated using the pulse information such as the number of pulse beats in a lifetime and measured pulse rates, the convenience of the portable terminal device is enhanced.

(3) Since the course of changes in pulse information is referred, a virtual life expectancy value of a practical level is calculated, which contributes to presenting useful health information.

Other objects, features, and advantages of the present invention will become apparent by referring to the attached drawings and the embodiments.

The life expectancy estimation apparatus, the portable terminal device, the life expectancy estimation method, and the life expectancy estimation program disclosed herein calculate a life expectancy by using pulse information. Since the life expectancy is calculated on the basis of the number of pulse beats in a lifetime while using the present pulse rate and the recorded past pulse rates, life expectancy information and message information regarding health maintenance can be obtained with a simple structure without requiring specialized knowledge or judgment. Thus, the present invention is useful.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiments of the present inventions have been described in detail, it should be understood that the various changes, substitutions, and alterations could be made hereto without departing from the spirit and scope of the invention.

What is claimed is:

1. A life expectancy estimation apparatus for estimating a predicted value of life expectancy by pulse information of a biological body, the life expectancy estimation apparatus comprising:
an obtaining operable to obtain the pulse information;
a storage for storing a record of pulse information of a biological body including a plurality of entries of pulse beat rates of the biological body over a period of time; and
a calculation unit operable to calculate an estimated value of life expectancy from the number of remaining pulse beats and the pulse beat rates, the number of the remaining pulse beats being estimated by subtracting the number of total pulse beats in the past estimated from the record of the pulse information from the predetermined estimated value of total pulse beats in a whole life time of the biological body.

2. The life expectancy estimation apparatus according to claim 1, further comprising a time keeping unit for keeping a time at which the pulse information is obtained, wherein the storage stores age information of the biological body and the time, and the calculation unit calculates the number of the remaining pulse beats by referring to the pulse information stored on the storage and the age information and/or the time.

3. The life expectancy estimation apparatus according to claim 1, further comprising a posting processor for posting the estimated value and/or message information in accordance with the estimated value.

4. The life expectancy estimation apparatus according to claim 1, wherein the obtaining unit obtains the pulse information by irradiating near-infrared light to a vein of the biological body and sensing the amount of light transmitted thorough the vein, the amount of light varying depending on the amount of the blood flow in the vein.

5. The life expectancy estimation apparatus according to claim 1, wherein the life expectancy estimation apparatus is a portable terminal device.

6. A method for estimating a predicted value of life expectancy by pulse information of a biological body, the method comprising:
obtaining the pulse information;
storing a record of pulse information of a biological body including a plurality of entries of pulse beat rates of the biological body over a period of time; and
calculating an estimated value of life expectancy from the number of remaining pulse beats and the pulse beat rates, the number of the remaining pulse beats being estimated by subtracting the number of total pulse beats in the past estimated from the record of the pulse information from the predetermined estimated value of total pulse beats in a whole life time of the biological body.

7. A computer readable recording medium that stores therein a life expectancy estimation program for estimating a predicted value of life expectancy by pulse information of a biological body, the life expectancy determining program performing a process comprising:
obtaining the pulse information;
storing a record of pulse information of a biological body including a plurality of entries of pulse beat rates of the biological body over a period of time; and
calculating an estimated value of life expectancy from the number of remaining pulse beats and the pulse beat rates, the number of the remaining pulse beats being estimated by subtracting the number of total pulse beats in the past estimated from the record of the pulse information from the predetermined estimated value of total pulse beats in a whole life time of the biological body.
